(19) United States
(12) Patent Application Publication
(10) Pub. No.: US 2021/0121244 A1
(43) Pub. Date: Apr. 29, 2021

(54) SYSTEMS AND METHODS FOR LOCATING PATIENT FEATURES

(71) Applicant: SHANGHAI UNITED IMAGING INTELLIGENCE CO., LTD., Shanghai (CN)

(72) Inventors: Arun Innanje, Lexington, MA (US); Ziyan Wu, Lexington, MA (US); Srikrishna Karanam, Brighton, MA (US)

(21) Appl. No.: 16/665,804

(22) Filed: Oct. 28, 2019

(51) Int. Cl. A61B 34/00; G06T 3/00; G06T 7/00

(52) U.S. Cl. CPC A61B 34/25; G06T 3/0068; G06T 7/0012; A61B 2034/2065; G06T 2207/20081; A61B 2034/252; G06T 7/97

(57) ABSTRACT

Methods and systems for locating one or more target features of a patient. For example, a computer-implemented method includes receiving a first input image; receiving a second input image; generating a first patient representation corresponding to the first input image; generating a second patient representation corresponding to the second input image; determining one or more first features corresponding to the first patient representation in a feature space; determining one or more second features corresponding to the second patient representation in the feature space; joining the one or more first features and the one or more second features into one or more joined features; determining one or more landmarks based at least in part on the one or more joined features; and providing a visual guidance for a medical procedure based at least in part on the information associated with the one or more landmarks.

### 1. BACKGROUND OF THE INVENTION

Certain embodiments of the present invention are directed to feature visualization. More particularly, some embodiments of the invention provide methods and systems for locating patient features. Merely by way of example, some embodiments of the invention have been applied to providing visual guidance for medical procedures. But it would be recognized that the invention has a much broader range of applicability.

Various ailment treatments involve having a physical examination followed by a diagnostic scan, such as an X-ray, CT, MR, PET, or SPECT scan. A medical staff or doctor often relies on analyzing the scan result to help diagnose the cause of one or more symptoms and determine a treatment plan. For treatment plans involving operation procedures such as surgery, radiation therapy, and other interventional treatment, a region of interest is generally determined with the help of the scan result. It is therefore, highly desirable to be able to determine information associated with the region of interest, such as location, size, and shape, with high accuracy and precision. As an example, for the administration of radiation therapy for a patient being treated for cancer, the location, shape, and size of a tumor would need to be determined, such as in terms of coordinates in a patient coordinate system. Any degree of mis-prediction of the region of interest is undesirable and may lead to costly errors such as damage or loss of healthy tissues. Localization of target tissues in the patient coordinate system is an essential step in many medical procedures and is proven to be a difficult problem to automate. As a result, many workflows rely on human inputs, such as inputs from experienced doctors. Some involve manually placing permanent tattoo around the region of interest and tracking the marked region using a monitoring system. Those manual and semi-automated methods are often resource-draining and prone to human error. Thus, systems and methods for locating patient features with high accuracy, precision, and optionally in real-time, are of great interest.

### 2. BRIEF SUMMARY OF THE INVENTION

Certain embodiments of the present invention are directed to feature visualization. More particularly, some embodiments of the invention provide methods and systems for locating patient features. Merely by way of example, some embodiments of the invention have been applied to providing visual guidance for medical procedures. But it would be recognized that the invention has a much broader range of applicability.

In various embodiments, a computer-implemented method for locating one or more target features of a patient includes: receiving a first input image; receiving a second input image; generating a first patient representation corresponding to the first input image; generating a second patient representation corresponding to the second input image; determining one or more first features corresponding to the first patient representation in a feature space; determining one or more second features corresponding to the second patient representation in the feature space; joining the one or more first features and the one or more second features into one or more joined features; determining one or more landmarks based at least in part on the one or more joined features; and providing a visual guidance for a medical procedure based at least in part on the information associated with the one or more landmarks. In certain examples, the computer-implemented method is performed by one or more processors.

In various embodiments, a system for locating one or more target features of a patient includes: an image receiving module configured to receive a first input image and receive a second input image; a representation generating module configured to generate a first patient representation corresponding to the first input image and generate a second patient representation corresponding to the second input image; a feature determining module configured to determine one or more first features corresponding to the first patient representation in a feature space and determine one or more second features corresponding to the second patient representation in the feature space; a feature joining module configured to join the one or more first features and the one or more second features into one or more joined features; a landmark determining module configured to determine one or more landmarks based at least in part on the one or more joined features; and a guidance providing module configured to provide a visual guidance based at least in part on the information associated with the one or more landmarks.

In various embodiments, a non-transitory computer-readable medium with instructions stored thereon, that when executed by a processor, perform the processes including: receiving a first input image; receiving a second input image; generating a first patient representation corresponding to the first medical image; generating a second patient representation corresponding to the second medical image; determining one or more first features corresponding to the first patient representation in a feature space; determining one or more second features corresponding to the second patient representation in the feature space; joining the one or more first features and the one or more second features into one or more joined features; determining one or more landmarks based at least in part on the one or more joined features; and providing a visual guidance for a medical procedure based at least in part on the information associated with the one or more landmarks.

Depending upon embodiment, one or more benefits may be achieved. These benefits and various additional objects, features and advantages of the present invention can be fully appreciated with reference to the detailed description and accompanying drawings that follow.

### 3. BRIEF DESCRIPTION OF THE DRAWINGS

FIG. 1 is a simplified diagram showing a system for locating one or more target features of a patient, according to some embodiments.

FIG. 2 is a simplified diagram showing a method for locating one or more target features of a patient, according to some embodiments.

FIG. 3 is a simplified diagram showing a method for training a machine learning model configured for locating one or more target features of a patient, according to some embodiments.

FIG. 4 is a simplified diagram showing a computing system, according to some embodiments.

FIG. 5 is a simplified diagram showing a neural network, according to some embodiments.

### 4. DETAILED DESCRIPTION OF THE INVENTION

Certain embodiments of the present invention are directed to feature visualization. More particularly, some embodiments of the invention provide methods and systems for locating patient features. Merely by way of example, some embodiments of the invention have been applied to providing visual guidance for medical procedures. But it would be recognized that the invention has a much broader range of applicability.

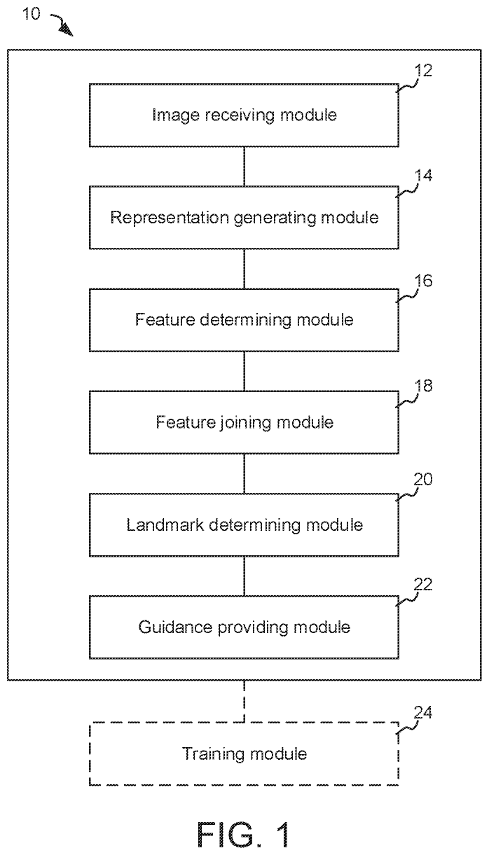

FIG. 1 is a simplified diagram showing a system for locating one or more target features of a patient, according to some embodiments. This diagram is merely an example, which should not unduly limit the scope of the claims. One of ordinary skill in the art would recognize many variations, alternatives, and modifications. In some examples, the system 10 includes an image receiving module 12, a representation generating module 14, a feature determining module 16, a feature joining module 18, a landmark determining module 20, and a guidance providing module 22. In certain examples, the system 10 further includes or is coupled to a training module 24. In various examples, the system 10 is a system for locating one or more target features (e.g., tissues, organs) of a patient. Although the above has been shown using a selected group of components, there can be many alternatives, modifications, and variations. For example, some of the components may be expanded and/or combined. Some components may be removed. Other components may be inserted to those noted above. Depending upon the embodiment, the arrangement of components may be interchanged with others replaced.

In various embodiments, the image receiving module 12 is configured to receive one or more images, such as one or more input images, one or more training images, and/or one or more patient images. In some examples, the one or more images includes a patient visual image obtained using a visual sensor, such as a RGB sensor, a RGBD sensor, a laser sensor, a FIR sensor, a NIR sensor, an X-ray sensor, or a lidar sensor. In various examples, the one or more images includes a scan image obtained using a medical scanner, such as an ultrasound scanner, an X-ray scanner, a MR scanner, a CT scanner, a PET scanner, a SPECT scanner, or a RGBD scanner. In certain examples, the patient visual image is two-dimensional and/or the scan image is three-dimensional. In some examples, the system 10 further includes an image acquiring module configured to acquire the patient visual image using a visual sensor and acquire the scan image using a medical scanner.

In various embodiments, the representation generating module 14 is configured to generate one or more patient representations, such as based at least in part on the one or more images. In some examples, the one or more patient representations includes a first patient representation corresponding to the patient visual image and a second patient representation corresponding to the scan image. In various examples, a patient representation includes an anatomical image, a kinematic model, a skeleton model, a surface model, a mesh model, and/or a point cloud. In certain examples, a patient representation includes information corresponding to one or more patient features. In certain embodiments, the representation generating module 14 is configured to generate the one or more patient representations by a machine learning model, such as a neural network, such as a deep neural network, such as a convolutional neural network.

In various embodiments, the feature determining module 16 is configured to determine one or more patient features for each patient representation of the one or more patient representations. In some examples, the feature determining module 16 is configured to determine one or more first patient features corresponding to the first patient representation in a feature space. In certain examples, the feature determining module 16 is configured to determine one or more second patient features corresponding to the second patient representation in a feature space. For example, the one or more first patient features and the one or more second patient features are in the same common feature space. In some examples, a feature space is referred to as a latent space. In various examples, the one or more patient features corresponding to a patient representation includes a pose, a surface feature, and/or an anatomical landmark (e.g., tissue, organ, foreign object). In certain examples, the feature determining module 16 is configured to determine one or more feature coordinates corresponding to each one or more patient features. For example, the feature determining module 16 is configured to determine one or more first feature coordinates corresponding to the one or more first patient features and determine one or more second feature coordinates corresponding to the one or more second patient features. In certain embodiments, the feature determining module 16 is configured to determine one or more patient features by a machine learning model, such as a neural network, such as a deep neural network, such as a convolutional neural network.

In various embodiments, the feature joining module 18 is configured to join a first feature in the feature space to a second feature in the feature space. In certain examples, the feature joining module 18 is configured to join a first patient feature corresponding to the first patient representation and the patient visual image to a second patient feature corresponding to the second patient representation and the scan image. In some examples, the feature joining module 18 is configured to join the one or more first patient features and the one or more second patient features into one or more joined patient features. In various examples, the feature joining module 18 is configured to match the one or more first patient features to the one or more second patient features. For example, the feature joining module 18 is configured to identify which of the second patient feature of the one or more second patient features does each of the first patient feature of the one or more first patient features corresponds to. In certain examples, the feature joining module 18 is configured to align the one or more first patient features to the one or more second patient features. For example, the feature joining module 18 is configured to transform the distribution of the one or more first patient features in the feature space relative to the one or more second patient features, such as via translational and/or rotational transformation, to align the one or more first patient features to the one or more second patient features. In various examples, the feature joining module 18 is configured to align the one or more first feature coordinates to the one or more second feature coordinates. In certain examples, one or more anchor features are used to guide the alignment. For example, the one or more anchor features included in both the one or more first patient features and the one or more second patient features are aligned substantially to the same coordinates in the feature space.

In various examples, the feature joining module 18 is configured to pair each first patient feature of the one or more first patient features to a second patient feature of the one or more second patient features. For example, the feature joining module 18 is configured to pair (e.g., link, combine, share) information corresponding to the first patient feature to information corresponding to the second patient feature. In certain examples, the paired information corresponding to a paired feature is used for minimizing information deviation of a common anatomical feature (e.g., a landmark) from images obtained via different imaging modalities. For example, pairing a first unpaired information, determined based on a patient visual image, to a second unpaired information, determined based on a scan image, generates a paired information for a target feature. In certain examples, the feature joining module 18 is configured to embed a common feature shared in multiple images obtained by multiple modalities (e.g., image acquisition devices) in the common feature space by assigning a joined coordinate to a joined patient feature in the common feature space based at least in part on information associated with the common feature from the multiple images. In some examples, the common feature space is shared across all different modalities. In certain examples, the common feature space is different for each pair of modalities. In certain embodiments, the feature joining module 18 is configured to join a first patient feature in the feature space to a second patient feature in the common feature space by a machine learning model, such as a neural network, such as a deep neural network, such as a convolutional neural network.

In various embodiments, the landmark determining module 20 is configured to determine one or more landmarks based at least in part on one or more joined patient features. For example, the one or more landmarks includes a patient tissue, an organ, or an anatomical structure. In certain examples, the landmark determining module 20 is configured to match each landmark with the reference medical imaging data of the patient. For example, the reference medical imaging data corresponds to the common feature space. In various examples, the landmark determining module 20 is configured to determine a landmark (e.g., an anatomical landmark) by identifying signature (e.g., shape, location) and/or feature representation shared across images obtained by different modalities. In some examples, the landmark determining module 20 is configured to map and/or interpolate the landmark onto a patient coordinate system and/or a display coordinate system. In certain examples, the landmark determining module 20 is configured to prepare the landmark for navigation and/or localization in a visual display having the patient coordinate system. In certain embodiments, the landmark determining module 20 is configured to determine one or more landmarks by a machine learning model, such as a neural network, such as a deep neural network, such as a convolutional neural network.

In various embodiments, the guidance providing module 22 is configured to provide a visual guidance based at least in part on the information associated with the one or more landmarks. For example, the information associated with the one or more landmarks includes a landmark name, a landmark coordinate, a landmark size, and/or a landmark property. In some examples, the guidance providing module 22 is configured to provide visual of the mapped and interpolated one or more landmarks in the patient coordinate system and/or the display coordinate system. In various examples, the guidance providing module 22 is configured to localize (e.g., zoom in, focus, position) a display region onto a target region based at least in part on a selected target landmark. For example, the target region spans the chest cavity when the selected target landmark is the heart. In certain examples, such as when the medical procedure is an interventional procedure, the guidance providing module 22 is configured to provide information associated with one or more targets of interest including a number of targets, one or more target coordinates, one or more target sizes, and/or one or more target shapes. In certain examples, such as when the medical procedure is a radiation therapy, the guidance providing module 22 is configured to provide information associated with a region of interest including a region size and/or a region shape. In various examples, the guidance providing module 22 is configured to provide the visual guidance to a visual display, such as a visual display observable, navigable, and/or localizable in an operating room.

In certain examples, the system 10 is configured to enable the guidance providing module 22 to provide real time or near real time update of information associated with the one or more landmarks, such as in response to manipulation of a patient (e.g., change of patient pose). For example, the image receiving module 12 is configured to continuously or intermittently receive (e.g., from the image acquiring module) new images corresponding to the patient from two or more modalities, the representation generating module 14 is configured to generate new patient representations based on the new images, the feature determining module 16 is configured to generate new patient features based on the new patient representations, the feature joining module 18 is configured to join one or more new patient features, the landmark determining module 20 is configured to determine one or more updated landmarks based on the one or more joined new patient features, and the guidance providing module 22 is configured to provide guidance including information associated with the one or more updated landmarks.

In various embodiments, the training module 24 is configured to improve system 10, such as the accuracy, precision, and/or speed of system 10 in providing information associated with one or more landmarks. In some examples, the training module 24 is configured to train the representation generating module 14, the feature determining module 16, the feature joining module 18, and/or the landmark determining module 20. For example, the training module 24 is configured to train a machine learning model used by one or more of the modules, such as a neural network, such as a deep neural network, such as a convolutional neural network. In certain examples, the training module 24 is configured to train the machine learning model by at least determining one or more losses between the one or more first patient features and the one or more second patient features and modifying one or more parameters of the machine learning model based at least in part on the one or more losses. In some examples, modifying the one or more parameters of the machine learning model based at least in part on the one or more losses includes modifying one or more parameters of the machine learning model to reduce (e.g., minimize) the one or more losses.

In certain embodiments, the system 10 is configured to automate the feature locating process by the use of one or more visual sensors and one or more medical scanners, matching and alignment of patient features, determination and localization of landmarks, and pairing and presenting of cross-referenced landmark coordinates. In some examples, the system 10 is configured to be utilized in radiation therapy to provide visual guidance, such as to localize a tumor or cancerous tissues to aid treatment with improved accuracy and precision. In various examples, the system 10 is configured to be utilized in interventional procedures to provide visual guidance, such as to localize one or more cysts in the patient to guide the surgical procedure. In certain examples, the system 10 is configured to utilize a projection technology such as augmented reality to overlay the landmark information (e.g., location, shape, size), determined by system 10, onto the patient, such as in real time, to guide the doctor throughout the medical procedure.

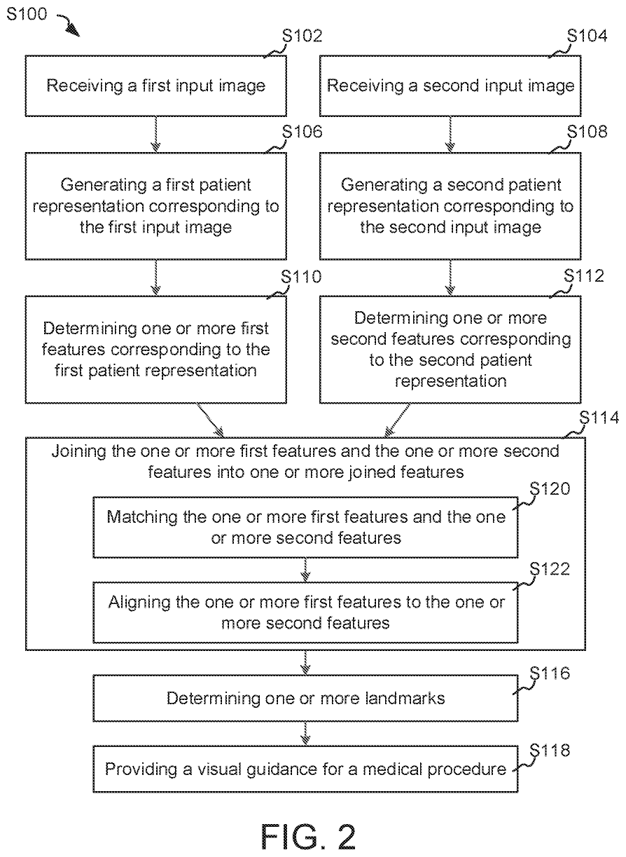

FIG. 2 is a simplified diagram showing a method for locating one or more target features of a patient, according to some embodiments. This diagram is merely an example, which should not unduly limit the scope of the claims. One of ordinary skill in the art would recognize many variations, alternatives, and modifications. In some examples, the method S100 includes a process S102 of receiving a first input image, a process S104 of receiving a second input image, a process S106 of generating a first patient representation, a process S108 of generating a second patient representation, a process S110 of determining one or more first features, a process S112 of determining one or more second features, a process S114 of j oining the one or more first features and the one or more second features, a process S116 of determining one or more landmarks, and a process S118 of providing a visual guidance for a medical procedure. In various examples, the method S100 is a method for locating one or more target features of a patient. In some examples, the method S100 is performed by one or more processors, such as using a machine learning model. Although the above has been shown using a selected group of processes for the method, there can be many alternatives, modifications, and variations. For example, some of the processes may be expanded and/or combined. Other processes may be inserted to those noted above. Some processes may be removed. Depending upon the embodiment, the sequence of processes may be interchanged with others replaced.

In various embodiments, the process S102 of receiving a first input image includes receiving a first input image obtained using a visual sensor, such as a RGB sensor, a RGBD sensor, a laser sensor, a FIR sensor, a NIR sensor, an X-ray sensor, or a lidar sensor. In certain examples, the first input image is two-dimensional. In various examples, the method S100 includes acquiring the first input image using a visual sensor.

In various embodiments, the process S104 of receiving a second input image includes receiving a second input image obtained using a medical scanner, such as an ultrasound scanner, an X-ray scanner, a MR scanner, a CT scanner, a PET scanner, a SPECT scanner, or a RGBD scanner. In certain examples, the second input image is three-dimensional. In various examples, the method S100 includes acquiring the second input image using a medical scanner.

In various embodiments, the process S106 of generating a first patient representation includes generating the first patient representation corresponding to the first input image. In various examples, the first patient representation includes an anatomical image, a kinematic model, a skeleton model, a surface model, a mesh model, and/or a point cloud. In certain examples, the first patient representation includes information corresponding to one or more first patient features. In certain embodiments, generating a first patient representation includes generating a first patient representation by a machine learning model, such as a neural network, such as a deep neural network, such as a convolutional neural network.

In various embodiments, the process S108 of generating a second patient representation includes generating the second patient representation corresponding to the second input image. In various examples, the second patient representation includes an anatomical image, a kinematic model, a skeleton model, a surface model, a mesh model, and/or a point cloud. In certain examples, the second patient representation includes information corresponding to one or more second patient features. In certain embodiments, generating a second patient representation includes generating a second patient representation by a machine learning model, such as a neural network, such as a deep neural network, such as a convolutional neural network.

In various embodiments, the process S110 of determining one or more first features includes determining one or more first features corresponding to the first patient representation, in a common feature space. In various examples, the one or more first features includes a pose, a surface feature, and/or an anatomical landmark (e.g., tissue, organ, foreign object). In some examples, determining one or more first features corresponding to the first patient representation includes determining one or more first coordinates (e.g., in the feature space) corresponding to the one or more first features. In certain embodiments, determining one or more first features includes determining one or more first features by a machine learning model, such as a neural network, such as a deep neural network, such as a convolutional neural network.

In various embodiments, the process S112 of determining one or more second features includes determining one or more second features corresponding to the second patient representation, in the common feature space. In various examples, the one or more second features includes a pose, a surface feature, and/or an anatomical landmark (e.g., tissue, organ, foreign object). In some examples, determining one or more second features corresponding to the second patient representation includes determining one or more second coordinates (e.g., in the feature space) corresponding to the one or more second features. In certain embodiments, determining one or more second features includes determining one or more second features by a machine learning model, such as a neural network, such as a deep neural network, such as a convolutional neural network.

In various embodiments, the process S114 of joining the one or more first features and the one or more second features includes joining the one or more first features and the one or more second features into one or more joined features. In some examples, joining the one or more first features and the one or more second features into one or more joined features includes the process S120 of matching the one or more first features to the one or more second features. For example, matching the one or more first features to the one or more second features includes identifying which of the second feature of the one or more second features does each of the first feature of the one or more first features corresponds to. In certain examples, joining the one or more first features to the one or more second features includes the process S122 of aligning the one or more first features to the one or more second features. For example, aligning the one or more first features to the one or more second features includes transforming the distribution of the one or more first features in the common feature space relative to the one or more second features, such as via translational and/or rotational transformation. In various examples, aligning the one or more first features to the one or more second features includes aligning the one or more first coordinates corresponding to the one or more first features to the one or more second coordinates corresponding to the one or more second features. In certain examples, aligning the one or more first features to the one or more second features includes using one or more anchor features as guidance. For example, the one or more anchor features included in both the one or more first features and the one or more second features are aligned substantially to the same coordinates in the common feature space.

In various examples, joining the one or more first features and the one or more second features includes pairing each first feature of the one or more first features to a second feature of the one or more second features. For example, pairing a first feature to a second feature includes pairing (e.g., linking, combining, sharing) information corresponding to the first feature to information corresponding to the second feature. In certain examples, the method S100 includes minimizing information deviation of a common anatomical feature (e.g., a landmark) from images obtained via different imaging modalities using the paired information corresponding to the common anatomical feature. In certain examples, joining the one or more first features and the one or more second features includes embedding a common feature shared in multiple images obtained by multiple modalities (e.g., image acquisition devices) in the common feature space. For example, embedding a common feature includes assigning a joined coordinate to a joined patient feature in the common feature space based at least in part on information associated with the common feature from the multiple images. In certain embodiments, joining the one or more first features and the one or more second features includes joining the one or more first features and the one or more second features by a machine learning model, such as a neural network, such as a deep neural network, such as a convolutional neural network.

In various embodiments, the process S116 of determining one or more landmarks includes determining one or more landmarks based at least in part on the one or more joined features. In some examples, the one or more landmarks includes a patient tissue, an organ, or an anatomical structure. In certain examples, determining one or more landmarks includes matching each landmark with the reference medical imaging data of the patient. For example, the reference medical imaging data corresponds to the common feature space. In various examples, determining one or more landmarks includes identifying one or more signatures (e.g., shape, location) and/or features shared across images obtained by different modalities. In certain embodiments, determining one or more landmarks includes determining one or more landmarks by a machine learning model, such as a neural network, such as a deep neural network, such as a convolutional neural network.

In various embodiments, the process S118 of providing a visual guidance for a medical procedure includes providing a visual guidance based at least in part on the information associated with the one or more landmarks. In some examples, the information associated with the one or more landmarks includes a landmark name, a landmark coordinate, a landmark size, and/or a landmark property. In various examples, providing a visual guidance for a medical procedure includes mapping and interpolating the one or more landmarks onto a patient coordinate system. In some examples, providing a visual guidance includes providing visual of one or more mapped and interpolated landmarks in a patient coordinate system and/or a display coordinate system. In various examples, providing a visual guidance includes localizing a display region onto a target region based at least in part on a selected target landmark. For example, the target region spans the chest cavity when the selected target landmark is the heart. In certain examples, such as when the medical procedure is an interventional procedure, providing a visual guidance includes providing information associated with one or more targets of interest including a number of targets, one or more target coordinates, one or more target sizes, and/or one or more target shapes. In certain examples, such as when the medical procedure is a radiation therapy, providing a visual guidance includes providing information associated with a region of interest including a region size and/or a region shape. In various examples, providing a visual guidance includes providing the visual guidance to a visual display, such as a visual display observable, navigable, and/or localizable in an operating room.

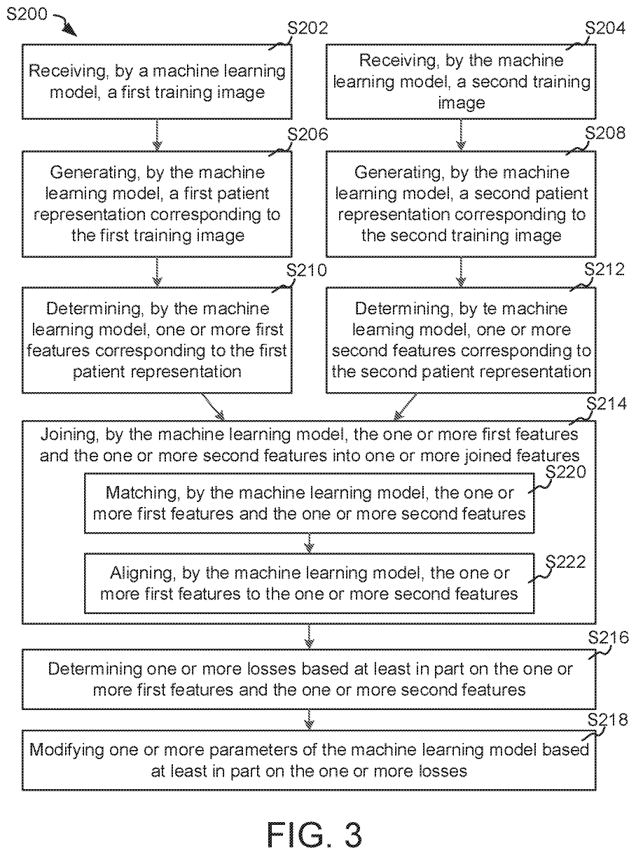

FIG. 3 is a simplified diagram showing a method for training a machine learning model configured for locating one or more target features of a patient, according to some embodiments. This diagram is merely an example, which should not unduly limit the scope of the claims. One of ordinary skill in the art would recognize many variations, alternatives, and modifications. In some examples, the method S200 includes a process S202 of receiving a first training image, a process S204 of receiving a second training image, a process S206 of generating a first patient representation, a process S208 of generating a second patient representation, a process S210 of determining one or more first features, a process S212 of determining one or more second features, a process S214 of joining the one or more first features and the one or more second features, a process S216 of determining one or more losses, and a process S218 of modifying one or more parameters of the machine learning model. In various examples, the machine learning model is a neural network, such as a deep neural network, such as a convolutional neural network. In certain examples, the machine learning model, such as once trained according to the method S200, is configured to be used by one or more processes of the method S100. Although the above has been shown using a selected group of processes for the method, there can be many alternatives, modifications, and variations. For example, some of the processes may be expanded and/or combined. Other processes may be inserted to those noted above. Some processes may be removed. Depending upon the embodiment, the sequence of processes may be interchanged with others replaced.

In various embodiments, the process S202 of receiving a first training image includes receiving a first training image obtained using a visual sensor, such as a RGB sensor, a RGBD sensor, a laser sensor, a FIR sensor, a NIR sensor, an X-ray sensor, or a lidar sensor. In certain examples, the first training image is two-dimensional.

In various embodiments, the process S204 of receiving a second training image includes receiving a second training image obtained using a medical scanner, such as an ultrasound scanner, an X-ray scanner, a MR scanner, a CT scanner, a PET scanner, a SPECT scanner, or a RGBD scanner. In certain examples, the second training image is three-dimensional.

In various embodiments, the process S206 of generating a first patient representation includes generating the first patient representation corresponding to the first training image. In various examples, the first patient representation includes an anatomical image, a kinematic model, a skeleton model, a surface model, a mesh model, and/or a point cloud. In certain examples, the first patient representation includes information corresponding to one or more first patient features. In certain embodiments, generating a first patient representation includes generating the first patient representation by the machine learning model.

In various embodiments, the process S208 of generating a second patient representation includes generating the second patient representation corresponding to the second training image. In various examples, the second patient representation includes an anatomical image, a kinematic model, a skeleton model, a surface model, a mesh model, and/or a point cloud. In certain examples, the second patient representation includes information corresponding to one or more second patient features. In certain embodiments, generating a second patient representation includes generating the second patient representation by the machine learning model.

In various embodiments, the process S210 of determining one or more first features includes determining one or more first features corresponding to the first patient representation, in a common feature space. In various examples, the one or more first features includes a pose, a surface feature, and/or an anatomical landmark (e.g., tissue, organ, foreign object). In some examples, determining one or more first features corresponding to the first patient representation includes determining one or more first coordinates (e.g., in the feature space) corresponding to the one or more first features. In certain embodiments, determining one or more first features includes determining one or more first features by the machine learning model.

In various embodiments, the process S212 of determining one or more second features includes determining one or more second features corresponding to the second patient representation, in the common feature space. In various examples, the one or more second features includes a pose, a surface feature, and/or an anatomical landmark (e.g., tissue, organ, foreign object). In some examples, determining one or more second features corresponding to the second patient representation includes determining one or more second coordinates (e.g., in the feature space) corresponding to the one or more second features. In certain embodiments, determining one or more second features includes determining one or more second features by the machine learning model.

In various embodiments, the process S214 of joining the one or more first features and the one or more second features includes joining the one or more first features and the one or more second features into one or more joined features. In some examples, joining the one or more first features and the one or more second features into one or more joined features includes a process S220 of matching the one or more first features to the one or more second features. For example, matching the one or more first features to the one or more second features includes identifying which of the second feature of the one or more second features does each of the first feature of the one or more first features corresponds to. In certain examples, joining the one or more first features to the one or more second features includes a process S222 of aligning the one or more first features to the one or more second features. For example, aligning the one or more first features to the one or more second features includes transforming the distribution of the one or more first features in the common feature space relative to the one or more second features, such as via translational and/or rotational transformation. In various examples, aligning the one or more first features to the one or more second features includes aligning the one or more first coordinates corresponding to the one or more first features to the one or more second coordinates corresponding to the one or more second features. In certain examples, aligning the one or more first features to the one or more second features includes using one or more anchor features as guide. For example, the one or more anchor features included in both the one or more first features and the one or more second features are aligned substantially to the same coordinates in the common feature space.

In various examples, the process S214 of joining the one or more first features and the one or more second features further includes pairing each first feature of the one or more first features to a second feature of the one or more second features. For example, pairing a first feature of the one or more first features to a second feature of the one or more second feature includes pairing (e.g., linking, combining, sharing) information corresponding to the first feature to information corresponding to the second feature. In certain examples, the method S200 includes minimizing information deviation of a common anatomical feature (e.g., a landmark) from images obtained via different imaging modalities using the paired information corresponding to the common anatomical feature. In certain examples, joining the one or more first features and the one or more second features includes embedding a common feature shared in multiple images obtained by multiple modalities (e.g., image acquisition devices) in the common feature space by assigning a joined coordinate to a joined patient feature in the common feature space based at least in part on information associated with the common feature from the multiple images. In certain embodiments, joining the one or more first features and the one or more second features includes joining the one or more first features and the one or more second features by the machine learning model.

In various embodiments, the process S216 of determining one or more losses includes determining one or more losses based at least in part on the one or more first features and the one or more second features. In certain examples, the process S216 of determining one or more losses includes determining one or more losses based at least in part on the one or more joined features. For example, the one or more losses corresponds to one or more deviations between the one or more first features and the one or more second features before and/or after joining, aligning, matching, and/or paring. In some examples, the one or more deviations includes one or more distances, such as one or more distances in the common feature space.

In various embodiments, the process S218 of modifying one or more parameters of the machine learning model includes modifying or changing one or more parameters of the machine learning model based at least in part on the one or more losses. In some examples, modifying one or more parameters of the machine learning model includes modifying one or more parameters of the machine learning model to reduce (e.g., minimize) the one or more losses. In certain examples, modifying one or more parameters of the machine learning model includes changing one or more weights and/or biases of the machine learning model, such as according to one or more gradients and/or a back-propagation process. In various embodiments, the process S218 of modifying one or more parameters of the machine learning model includes repeating one or more of processes S202, S204, S206, S208, S210, S212, S214, S216, and S218.

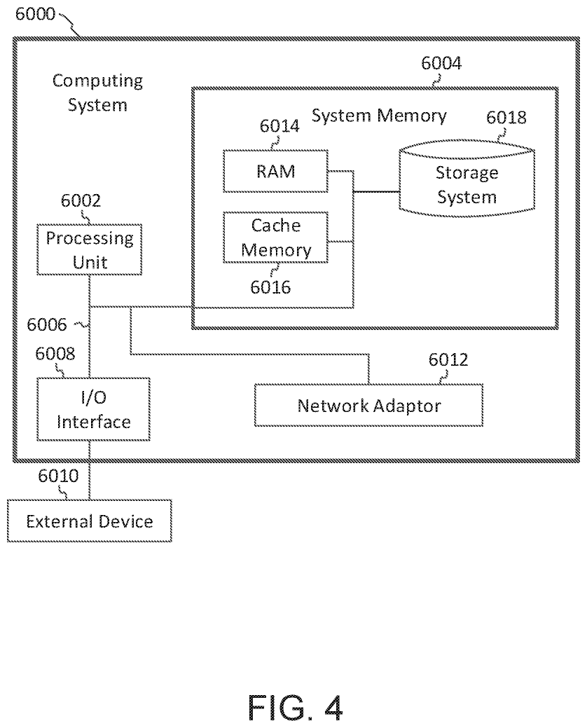

FIG. 4 is a simplified diagram showing a computing system, according to some embodiments. This diagram is merely an example, which should not unduly limit the scope of the claims. One of ordinary skill in the art would recognize many variations, alternatives, and modifications. In certain examples, the computing system 6000 is a general-purpose computing device. In some examples, the computing system 6000 includes one or more processing units 6002 (e.g., one or more processors), one or more system memories 6004, one or more buses 6006, one or more input/output (I/O) interfaces 6008, and/or one or more network adapters 6012. In certain examples, the one or more buses 6006 connect various system components including, for example, the one or more system memories 6004, the one or more processing units 6002, the one or more input/output (I/O) interfaces 6008, and/or the one or more network adapters 6012. Although the above has been shown using a selected group of components for the computing system, there can be many alternatives, modifications, and variations. For example, some of the components may be expanded and/or combined. Other components may be inserted to those noted above. Some components may be removed. Depending upon the embodiment, the arrangement of components may be interchanged with others replaced.

In certain examples, the computing system 6000 is a computer (e.g., a server computer, a client computer), a smartphone, a tablet, or a wearable device. In some examples, some or all processes (e.g., steps) of the method S100 and/or the method S200 are performed by the computing system 6000. In certain examples, some or all processes (e.g., steps) of the method S100 and/or the method S200 are performed by the one or more processing units 6002 directed by one or more codes. For example, the one or more codes are stored in the one or more system memories 6004 (e.g., one or more non-transitory computer-readable media), and are readable by the computing system 6000 (e.g., readable by the one or more processing units 6002). In various examples, the one or more system memories 6004 include one or more computer-readable media in the form of volatile memory, such as a random-access memory (RAM) 6014, a cache memory 6016, and/or a storage system 6018 (e.g., a floppy disk, a CD-ROM, and/or a DVD-ROM).

In some examples, the one or more input/output (I/O) interfaces 6008 of the computing system 6000 is configured to be in communication with one or more external devices 6010 (e.g., a keyboard, a pointing device, and/or a display). In certain examples, the one or more network adapters 6012 of the computing system 6000 is configured to communicate with one or more networks (e.g., a local area network (LAN), a wide area network (WAN), and/or a public network (e.g., the Internet)). In various examples, additional hardware and/or software modules are utilized in connection with the computing system 6000, such as one or more micro-codes and/or one or more device drivers.

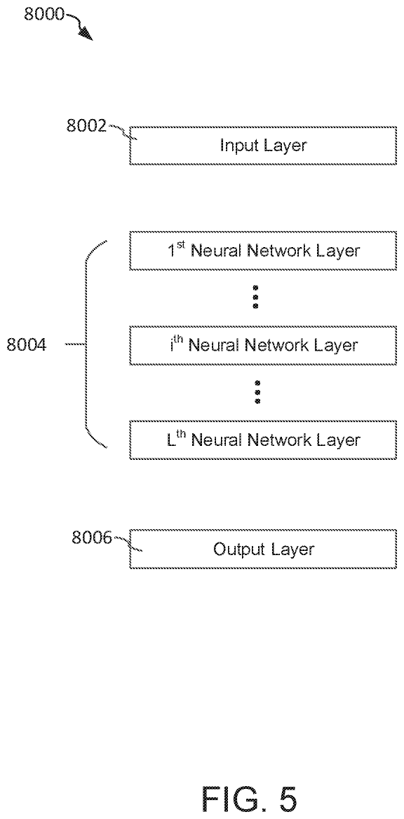

FIG. 5 is a simplified diagram showing a neural network, according to certain embodiments. This diagram is merely an example, which should not unduly limit the scope of the claims. One of ordinary skill in the art would recognize many variations, alternatives, and modifications. The neural network 8000 is an artificial neural network. In some examples, the neural network 8000 includes an input layer 8002, one or more hidden layers 8004, and an output layer 8006. For example, the one or more hidden layers 8004 includes L number of neural network layers, which include a 1^{st} neural network layer, . . . , an i^{th} neural network layer, . . . and an L^{th} neural network layer, where L is a positive integer and i is an integer that is larger than or equal to 1 and smaller than or equal to L. Although the above has been shown using a selected group of components for the neural network, there can be many alternatives, modifications, and variations. For example, some of the components may be expanded and/or combined. Other components may be inserted to those noted above. Some components may be removed. Depending upon the embodiment, the arrangement of components may be interchanged with others replaced.

In some examples, some or all processes (e.g., steps) of the method S100 and/or the method S200 are performed by the neural network 8000 (e.g., using the computing system 6000). In certain examples, some or all processes (e.g., steps) of the method S100 and/or the method S200 are performed by the one or more processing units 6002 directed by one or more codes that implement the neural network 8000. For example, the one or more codes for the neural network 8000 are stored in the one or more system memories 6004 (e.g., one or more non-transitory computer-readable media), and are readable by the computing system 6000 such as by the one or more processing units 6002.

In certain examples, the neural network 8000 is a deep neural network (e.g., a convolutional neural network). In some examples, each neural network layer of the one or more hidden layers 8004 includes multiple sublayers. As an example, the i^{th} neural network layer includes a convolutional layer, an activation layer, and a pooling layer. For example, the convolutional layer is configured to perform feature extraction on an input (e.g., received by the input layer or from a previous neural network layer), the activation layer is configured to apply a nonlinear activation function (e.g., a ReLU function) to the output of the convolutional layer, and the pooling layer is configured to compress (e.g., to down-sample, such as by performing max pooling or average pooling) the output of the activation layer. As an example, the output layer 8006 includes one or more fully connected layers.

As discussed above and further emphasized here, FIG. 5 is merely an example, which should not unduly limit the scope of the claims. One of ordinary skill in the art would recognize many variations, alternatives, and modifications. For example, the neural network 8000 is replaced by an algorithm that is not an artificial neural network. As an example, the neural network 8000 is replaced by a machine learning model that is not an artificial neural network.

In various embodiments, a computer-implemented method for locating one or more target features of a patient includes: receiving a first input image; receiving a second input image; generating a first patient representation corresponding to the first input image; generating a second patient representation corresponding to the second input image; determining one or more first features corresponding to the first patient representation in a feature space; determining one or more second features corresponding to the second patient representation in the feature space; joining the one or more first features and the one or more second features into one or more joined features; determining one or more landmarks based at least in part on the one or more joined features; and providing a visual guidance for a medical procedure based at least in part on the information associated with the one or more landmarks. In certain examples, the computer-implemented method is performed by one or more processors. In some examples, the computer-implemented method is implemented according to the method S100 of FIG. 2 and/or the method S200 of FIG. 3. In certain examples, the method is implemented by the system 10 of FIG. 1.

In some embodiments, the computer-implemented method further includes acquiring the first input image using a visual sensor and acquiring the second input image using a medical scanner.

In some embodiments, the visual sensor includes a RGB sensor, a RGBD sensor, a laser sensor, a FIR sensor, a NIR sensor, an X-ray sensor, and/or a lidar sensor.

In some embodiments, the medical scanner includes an ultrasound scanner, an X-ray scanner, a MR scanner, a CT scanner, a PET scanner, a SPECT scanner, and/or a RGBD scanner.

In some embodiments, the first input image is two-dimensional, and/or the second input image is three-dimensional.

In some embodiments, the first patient representation includes an anatomical image, a kinematic model, a skeleton model, a surface model, a mesh model, and/or a point cloud. In certain examples, the second patient representation includes an anatomical image, a kinematic model, a skeleton model, a surface model, a mesh model, a point cloud, and/or a three-dimensional volume.

In some embodiments, the one or more first features includes a pose, a surface, and/or an anatomical landmark. In certain examples, the one or more second features includes a pose, a surface, and/or an anatomical landmark.

In some embodiments, joining the one or more first features and the one or more second features into one or more joined features includes matching the one or more first features to the one or more second features and/or aligning the one or more first features to the one or more second features.

In some embodiments, matching the one or more first features to the one or more second features includes pairing each first feature of the one or more first features to a second feature of the one or more second features.

In some embodiments, determining one or more first features corresponding to the first patient representation in a feature space includes determining one or more first coordinates corresponding to the one or more first features. In certain examples, determining one or more second features corresponding to the second patient representation in the feature space includes determining one or more second coordinates corresponding to the one or more second features. In various examples, aligning the one or more first features to the one or more second features includes aligning the one or more first coordinates to the one or more second coordinates.

In some embodiments, the information associated with the one or more landmarks includes a landmark name, a landmark coordinate, a landmark size, and/or a landmark property.

In some embodiments, providing a visual guidance for a medical procedure includes localizing a display region onto a target region based at least in part on a selected target landmark.

In some embodiments, providing a visual guidance for a medical procedure includes mapping and interpolating the one or more landmarks onto a patient coordinate system.

In some embodiments, the medical procedure is an interventional procedure. In certain examples, providing a visual guidance for a medical procedure includes providing information associated with one or more targets of interest. In various examples, the information includes a number of targets, one or more target coordinates, one or more target sizes, and/or one or more target shapes.

In some embodiments, the medical procedure is a radiation therapy. In certain examples, providing a visual guidance for a medical procedure includes providing information associated with a region of interest. In various examples, the information includes a region size and/or a region shape.

In some embodiments, the computer-implemented method is performed by one or more processors using a machine learning model.

In some embodiments, the computer-implemented method further includes training the machine learning model by at least determining one or more losses between the one or more first features and the one or more second features and modifying one or more parameters of the machine learning model based at least in part on the one or more losses.

In some embodiments, modifying one or more parameters of the machine learning model based at least in part on the one or more losses includes modifying one or more parameters of the machine learning model to reduce the one or more losses.

In various embodiments, a system for locating one or more target features of a patient includes: an image receiving module configured to receive a first input image and receive a second input image; a representation generating module configured to generate a first patient representation corresponding to the first input image and generate a second patient representation corresponding to the second input image; a feature determining module configured to determine one or more first features corresponding to the first patient representation in a feature space and determine one or more second features corresponding to the second patient representation in the feature space; a feature joining module configured to join the one or more first features and the one or more second features into one or more joined features; a landmark determining module configured to determine one or more landmarks based at least in part on the one or more joined features; and a guidance providing module configured to provide a visual guidance based at least in part on the information associated with the one or more landmarks. In some examples, the system is implemented according to the system 10 of FIG. 1 and/or configured to perform the method S100 of FIG. 2 and/or the method S200 of FIG. 3.

In some embodiments, the system further includes an image acquiring module configured to acquire the first input image using a visual sensor and acquire the second input image using a medical scanner.

In some embodiments, the visual sensor includes a RGB sensor, a RGBD sensor, a laser sensor, a FIR sensor, a NIR sensor, an X-ray sensor, and/or a lidar sensor.

In some embodiments, the medical scanner includes an ultrasound scanner, an X-ray scanner, a MR scanner, a CT scanner, a PET scanner, a SPECT scanner, and/or a RGBD scanner.

In some embodiments, the first input image is two-dimensional, and/or the second input image is three-dimensional.

In some embodiments, the first patient representation includes an anatomical image, a kinematic model, a skeleton model, a surface model, a mesh model, and/or a point cloud. In certain examples, the second patient representation includes an anatomical image, a kinematic model, a skeleton model, a surface model, a mesh model, a point cloud, and/or a three-dimensional volume.

In some embodiments, the one or more first features includes a pose, a surface, and/or an anatomical landmark. In certain examples, the one or more second features includes a pose, a surface, and/or an anatomical landmark.

In some embodiments, the feature joining module is further configured to match the one or more first features to the one or more second features and/or align the one or more first features to the one or more second features.

In some embodiments, the feature joining module is further configured to pair each first feature of the one or more first features to a second feature of the one or more second features.

In some embodiments, the feature determining module is further configured to determine one or more first coordinates corresponding to the one or more first features and determine one or more second coordinates corresponding to the one or more second features. In various examples, the feature joining module is further configured to align the one or more first coordinates to the one or more second coordinates.

In some embodiments, the information associated with the one or more landmarks includes a landmark name, a landmark coordinate, a landmark size, and/or a landmark property.

In some embodiments, the guidance providing module is further configured to localize a display region onto a target region based at least in part on a selected target landmark.

In some embodiments, the guidance providing module is further configured to map and interpolate the one or more landmarks onto a patient coordinate system.

In some embodiments, the medical procedure is an interventional procedure. In certain examples, the guidance providing module is further configured to provide information associated with one or more targets of interest. In various examples, the information includes a number of targets, one or more target coordinates, one or more target sizes, and/or one or more target shapes.

In some embodiments, the medical procedure is a radiation therapy. In certain examples, the guidance providing module is further configured to provide information associated with a region of interest. In various examples, the information includes a region size and/or a region shape.

In some embodiments, the system uses a machine learning model.

In various embodiments, a non-transitory computer-readable medium with instructions stored thereon, that when executed by a processor, causes the processor to perform one or more processes including: receiving a first input image; receiving a second input image; generating a first patient representation corresponding to the first medical image; generating a second patient representation corresponding to the second medical image; determining one or more first features corresponding to the first patient representation in a feature space; determining one or more second features corresponding to the second patient representation in the feature space; joining the one or more first features and the one or more second features into one or more joined features; determining one or more landmarks based at least in part on the one or more joined features; and providing a visual guidance for a medical procedure based at least in part on the information associated with the one or more landmarks. In some examples, the non-transitory computer-readable medium with instructions stored thereon is implemented according to the method S100 of FIG. 2, and/or by the system 10 (e.g., a terminal) of FIG. 1.

In some embodiments, the non-transitory computer-readable medium, that when executed by a processor, further causes the processor to perform: acquiring the first input image using a visual sensor and acquiring the second input image using a medical scanner.

In some embodiments, the visual sensor includes a RGB sensor, a RGBD sensor, a laser sensor, a FIR sensor, a NIR sensor, an X-ray sensor, and/or a lidar sensor.

In some embodiments, the medical scanner includes an ultrasound scanner, an X-ray scanner, a MR scanner, a CT scanner, a PET scanner, a SPECT scanner, and/or a RGBD scanner.

In some embodiments, the first input image is two-dimensional, and/or the second input image is three-dimensional.

In some embodiments, the first patient representation includes an anatomical image, a kinematic model, a skeleton model, a surface model, a mesh model, and/or a point cloud. In certain examples, the second patient representation includes an anatomical image, a kinematic model, a skeleton model, a surface model, a mesh model, a point cloud, and/or a three-dimensional volume.

In some embodiments, the one or more first features includes a pose, a surface, and/or an anatomical landmark. In certain examples, the one or more second features includes a pose, a surface, and/or an anatomical landmark.

In some embodiments, the non-transitory computer-readable medium, that when executed by a processor, further causes the processor to perform: matching the one or more first features to the one or more second features and/or aligning the one or more first features to the one or more second features.

In some embodiments, the non-transitory computer-readable medium, that when executed by a processor, further causes the processor to perform: pairing each first feature of the one or more first features to a second feature of the one or more second features.

In some embodiments, the non-transitory computer-readable medium, that when executed by a processor, further causes the processor to perform: determining one or more first coordinates corresponding to the one or more first features, determining one or more second coordinates corresponding to the one or more second features, and aligning the one or more first coordinates to the one or more second coordinates.

In some embodiments, the information associated with the one or more landmarks includes a landmark name, a landmark coordinate, a landmark size, and/or a landmark property.

In some embodiments, the non-transitory computer-readable medium, that when executed by a processor, further causes the processor to perform: localizing a display region onto a target region based at least in part on a selected target landmark.

In some embodiments, the non-transitory computer-readable medium, that when executed by a processor, further causes the processor to perform: mapping and interpolating the one or more landmarks onto a patient coordinate system.

In some embodiments, the medical procedure is an interventional procedure. In certain examples, the non-transitory computer-readable medium, that when executed by a processor, further causes the processor to perform: providing information associated with one or more targets of interest. In various examples, the information includes a number of targets, one or more target coordinates, one or more target sizes, and/or one or more target shapes.

In some embodiments, the medical procedure is a radiation therapy. In certain examples, the non-transitory computer-readable medium, that when executed by a processor, further causes the processor to perform: providing information associated with a region of interest. In various examples, the information includes a region size and/or a region shape.

For example, some or all components of various embodiments of the present invention each are, individually and/or in combination with at least another component, implemented using one or more software components, one or more hardware components, and/or one or more combinations of software and hardware components. In another example, some or all components of various embodiments of the present invention each are, individually and/or in combination with at least another component, implemented in one or more circuits, such as one or more analog circuits and/or one or more digital circuits. In yet another example, while the embodiments described above refer to particular features, the scope of the present invention also includes embodiments having different combinations of features and embodiments that do not include all of the described features. In yet another example, various embodiments and/or examples of the present invention can be combined.

Additionally, the methods and systems described herein may be implemented on many different types of processing devices by program code including program instructions that are executable by the device processing subsystem. The software program instructions may include source code, object code, machine code, or any other stored data that is operable to cause a processing system to perform the methods and operations described herein. Other implementations may also be used, however, such as firmware or even appropriately designed hardware configured to perform the methods and systems described herein.

The systems' and methods' data (e.g., associations, mappings, data input, data output, intermediate data results, final data results, etc.) may be stored and implemented in one or more different types of computer-implemented data stores, such as different types of storage devices and programming constructs (e.g., RAM, ROM, EEPROM, Flash memory, flat files, databases, programming data structures, programming variables, IF-THEN (or similar type) statement constructs, application programming interface, etc.). It is noted that data structures describe formats for use in organizing and storing data in databases, programs, memory, or other computer-readable media for use by a computer program.

The systems and methods may be provided on many different types of computer-readable media including computer storage mechanisms (e.g., CD-ROM, diskette, RAM, flash memory, computer's hard drive, DVD, etc.) that contain instructions (e.g., software) for use in execution by a processor to perform the methods' operations and implement the systems described herein. The computer components, software modules, functions, data stores and data structures described herein may be connected directly or indirectly to each other in order to allow the flow of data needed for their operations. It is also noted that a module or processor includes a unit of code that performs a software operation and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

The computing system can include client devices and servers. A client device and server are generally remote from each other and typically interact through a communication network. The relationship of client device and server arises by virtue of computer programs running on the respective computers and having a client device-server relationship to each other.

This specification contains many specifics for particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations, one or more features from a combination can in some cases be removed from the combination, and a combination may, for example, be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Although specific embodiments of the present invention have been described, it will be understood by those of skill in the art that there are other embodiments that are equivalent to the described embodiments. Accordingly, it is to be understood that the invention is not to be limited by the specific illustrated embodiments.

What is claimed is:

1. A computer-implemented method for locating one or more target features of a patient, the method comprising:
receiving a first input image;
receiving a second input image;
generating a first patient representation corresponding to the first input image;
generating a second patient representation corresponding to the second input image;
determining one or more first features corresponding to the first patient representation in a feature space;
determining one or more second features corresponding to the second patient representation in the feature space;
joining the one or more first features and the one or more second features into one or more joined features;
determining one or more landmarks based at least in part on the one or more joined features; and
providing a visual guidance for a medical procedure based at least in part on the information associated with the one or more landmarks;
wherein the computer-implemented method is performed by one or more processors.

2. The computer-implemented method of claim 1, further comprising:
acquiring the first input image using a visual sensor; and
acquiring the second input image using a medical scanner.

3. The computer-implemented method of claim 2, wherein the visual sensor includes at least one of a RGB sensor, a RGBD sensor, a laser sensor, a FIR sensor, a NIR sensor, an X-ray sensor, and a lidar sensor.

4. The computer-implemented method of claim 2, wherein the medical scanner includes at least one of an ultrasound scanner, an X-ray scanner, a MR scanner, a CT scanner, a PET scanner, a SPECT scanner, and a RGBD scanner.

5. The computer-implemented method of claim 1, wherein:
the first input image is two-dimensional; and
the second input image is three-dimensional.

6. The computer-implemented method of claim 1, wherein:
the first patient representation includes one selected from an anatomical image, a kinematic model, a skeleton model, a surface model, a mesh model, and a point cloud; and
the second patient representation includes one selected from an anatomical image, a kinematic model, a skeleton model, a surface model, a mesh model, a point cloud, and a three-dimensional volume.

7. The computer-implemented method of claim 1, wherein:
the one or more first features includes one selected from a pose, a surface, and an anatomical landmark; and
the one or more second features includes one selected from a pose, a surface, and an anatomical landmark.

8. The computer-implemented method of claim 1, wherein the joining the one or more first features and the one or more second features into one or more joined features includes:
matching the one or more first features to the one or more second features; and
aligning the one or more first features to the one or more second features.

9. The computer-implemented method of claim 8, wherein the matching the one or more first features to the one or more second features includes pairing each first feature of the one or more first features to a second feature of the one or more second features.

10. The computer-implemented method of claim 8, wherein:
determining one or more first features corresponding to the first patient representation in a feature space includes determining one or more first coordinates corresponding to the one or more first features;
determining one or more second features corresponding to the second patient representation in the feature space includes determining one or more second coordinates corresponding to the one or more second features; and
aligning the one or more first features to the one or more second features includes aligning the one or more first coordinates to the one or more second coordinates.

11. The computer-implemented method of claim 1, wherein the information associated with the one or more landmarks includes one of landmark name, landmark coordinate, landmark size, and landmark property.

12. The computer-implemented method of claim 1, wherein the providing a visual guidance for a medical procedure includes localizing a display region onto a target region based at least in part on a selected target landmark.

13. The computer-implemented method of claim 1, wherein the providing a visual guidance for a medical procedure includes mapping and interpolating the one or more landmarks onto a patient coordinate system.

14. The computer-implemented method of claim 1, wherein:
the medical procedure is an interventional procedure; and
the providing a visual guidance for a medical procedure includes providing information associated with one or more targets of interest, the information includes a number of targets, one or more target coordinates, one or more target sizes, or one or more target shapes.

15. The computer-implemented method of claim 1, wherein:
the medical procedure is a radiation therapy; and
the providing a visual guidance for a medical procedure includes providing information associated with a region of interest; the information includes a region size or a region shape.

16. The computer-implemented method of claim 1, wherein the computer-implemented method is performed by one or more processors using a machine learning model.

17. The computer-implemented method of claim 16, further comprising training the machine learning model by at least:
determining one or more losses between the one or more first features and the one or more second features; and
modifying one or more parameters of the machine learning model based at least in part on the one or more losses.

18. The computer-implemented method of claim 17, wherein modifying one or more parameters of the machine learning model based at least in part on the one or more losses includes:
modifying one or more parameters of the machine learning model to reduce the one or more losses.

19. A system for locating one or more target features of a patient, the system comprising:
an image receiving module configured to:
receive a first input image; and
receive a second input image;
a representation generating module configured to:
generate a first patient representation corresponding to the first input image; and
generate a second patient representation corresponding to the second input image;
a feature determining module configured to:
determine one or more first features corresponding to the first patient representation in a feature space; and
determine one or more second features corresponding to the second patient representation in the feature space;
a feature joining module configured to join the one or more first features and the one or more second features into one or more joined features;
a landmark determining module configured to determine one or more landmarks based at least in part on the one or more joined features; and
a guidance providing module configured to provide a visual guidance based at least in part on the information associated with the one or more landmarks.

20. A non-transitory computer-readable medium with instructions stored thereon, that when executed by a processor, causes the processor to perform one or more processes including:
receiving a first input image;
receiving a second input image;
generating a first patient representation corresponding to the first medical image;
generating a second patient representation corresponding to the second medical image;
determining one or more first features corresponding to the first patient representation in a feature space;
determining one or more second features corresponding to the second patient representation in the feature space;
joining the one or more first features and the one or more second features into one or more joined features;
determining one or more landmarks based at least in part on the one or more joined features; and
providing a visual guidance for a medical procedure based at least in part on the information associated with the one or more landmarks.
